(19) United States
(12) Patent Application Publication
(10) Pub. No.: US 2004/0162338 A1
(43) Pub. Date: Aug. 19, 2004

(54) Flavanols and procyanidins promote homeostasis

(76) Inventor: Harold H. Schmitz, Bethesda, MD (US)

Correspondence Address: NADA JAIN, P.C., 560 White Plains Road, Suite 460, Tarrytown, NY, 10591, US

(21) Appl. No.: 10/725,805

(22) Filed: Dec. 2, 2003

Related U.S. Application Data
(60) Provisional application No. 60/430,304, filed on Dec. 2, 2002.
(60) Provisional application No. 60/436,135, filed on Dec. 23, 2002.
(60) Provisional application No. 60/436,395, filed on Dec. 24, 2002.
(60) Provisional application No. 60/436,879, filed on Dec. 27, 2002.

(51) Int. Cl. A61K 31/7048; A61K 31/353; G01N 33/48; G01N 33/50; G06F 19/00

(52) U.S. Cl. 514/456; 702/19; 514/27

(57) ABSTRACT

The invention encompasses a screening method for identifying cytokine responsiveness of a human or a veterinary animal and products and assays for use therein comprising flavanols, procyanidins and/or derivatives thereof, or mixtures thereof, a method for diagnosing a cytokine phenotype in a human or a veterinary animal; methods for identifying a subject at risk of a condition associated with an inflammatory and/or immunomodulating pathway; methods for identifying a dietary and/or a pharmaceutical intervention to modulate a condition associated with an inflammatory and/or immunomodulating pathway; and the methods of prophylactic or therapeutic treatment of humans or veterinary animals with selected flavanols, procyanidins and/or derivatives thereof, or mixtures thereof.

This application claims the benefit, under 35 USC Section 119, of the U.S. Provisional Appl. Nos. 60/430,304 filed Dec. 2, 2002; 60/436,135 filed Dec. 23, 2002; 60/436,395 filed Dec. 24, 2002; and 60/436,879 filed Dec. 27, 2002, the disclosures of which are hereby incorporated herein by reference.

### FIELD OF THE INVENTION

The invention relates to a screening method for identifying cytokine responsiveness of a human or a veterinary animal and products and assays for use therein comprising flavanols and procyanidins, or mixtures thereof, a method for diagnosing a cytokine phenotype in a human or a veterinary animal; methods for identifying a subject at risk of a condition associated with an inflammatory and/or immunomodulating pathway; methods for identifying a dietary and/or a pharmaceutical intervention to modulate a condition associated with an inflammatory and/or immunomodulating pathway; and the methods of prophylactic or therapeutic treatment of humans or veterinary animals with selected flavanols and procyanidins, or mixtures thereof.

### BACKGROUND

Flavanols and procyanidins have demonstrated the potential to modulate a wide variety of factors associated with vascular health. This includes antioxidant actions (Lotito LB et al., Biochem Biophys Res Commun 2000;276:945-951, Arteel and Sies, FEBS Lett 1999;462:167-170; Sanbongi et al., Cell Immunol 1997;177:s 129-136), modulation of cytokine production (Mao et al., J Medicinal Foods 2002;5:17-22; Mao et al., Life Sciences 2000;66:1377-1386; Mao et al., J Medicinal Foods 2000;3:107-114), modulation of eicosanoids and NO and peroxynitrate levels and anti-inflammatory characteristics generally (Osakabe et al., Biosci Biotechnol Biochem 1998;62:1535-1538; Arteel and Sies, FEBS Lett 1999;462:167-170). Other beneficial effects of flavanols and procyanidins, for example, as anti-platelet and anti-microbial agents, and for cancer treatment have also been shown (U.S. Pat. Nos. 5,554,645 and 6,297,273).

TGF-β1 is a potent modulator of the cardiovascular system, thus considerable research has been devoted to the manipulation of its production and activity for therapeutic purposes. A variety of agents have been suggested to augment the production of TGF-β1. Metcalfe et al. suggested that tamoxifen reduced the formation of lipid lesions, in part by elevating circulating concentrations of TGF-β1 in mice subjected to a high-fat diet (Densem et al., J Heart Lung Trans 2000;19:551-556); while consistent with this, Djurovic et al reported that postmenopausal women undergoing hormone replacement therapy showed increased plasma concentrations of TGF-β1, suggesting a possible avenue to reduce the risk of cardiovascular disease (Grainger et al., Nat Med 1995; 1:1067-1073). The discovery of antagonists for TGF-β1 might be valuable in the treatment of fibrotic diseases. Decorin, a natural inhibitor of TGF-β1, has been used to successfully suppress TGF-β1-mediated tissue fibrosis in the rat kidney (Isaka et al., Nat Med 1996;2:418-423). In addition, resveratrol, a dietary plant polyphenol, was reported to have a protective effect against dysfunctions in vascular smooth muscle cells, in part due to its ability to inhibit TGF-β1 MRNA (Mizutani et al., Biochem Biophys Res Comm 2000;274:61-67). Given the importance of TGF-β1, additional approaches for regulating its levels in the body of a human or veterinary animal are needed.

Applicant has now surprisingly discovered that flavanols and procyanidins, and mixtures thereof, have the ability to promote cytokine homeostasis, including TGF-β1 homeostasis, and may be used as a screening tool to identify or phenotype cytokine responsiveness, including TGF-β1 responsiveness, in a subject. This allows physicians, clinicians, veterinarians and nutritionists to identify a subject, a human or a veterinary animal, as being at low or high risk for conditions associated with inflammatory and immunomodulating pathways, such as cardiovascular disease, arthritis, and cancer, and to design an appropriate dietary and/or a pharmaceutical intervention depending on that subject's phenotype.

### SUMMARY OF THE INVENTION

The invention relates to screening, diagnostic, prophylactic, therapeutic and nutritional applications of flavanols and/or procyanidins, and mixtures thereof, for diagnosis, prevention and/or treatment of conditions associated with inflammatory and/or immunomodulating pathways. The invention further relates to design of customized pharmaceutical and/or dietary regimens (i.e., interventions) for a human or a veterinary animal to address their health issues identified through the screening methodology described herein.

In one aspect, screening methodology and assays for identifying cytokine responsiveness in a human or a veterinary animal using flavanols and procyanidins, and mixtures thereof, are provided. Diagnostic assays for determining ex vivo baseline cytokine levels in body samples of humans or veterinary animals are also within the scope of the invention.

In another aspect, the invention relates to a method of designing a dietary and/or a pharmaceutical regimen (i.e., intervention) for a human or a veterinary animal depending on the human's or animal's phenotype (i.e., cytokine responsiveness), which regimen is effective at preventing or treating the health conditions diagnosed via the screening methodology and assays. In some embodiments, the regimen may comprise administration of a flavanol and/or a procyanidin, or any mixture thereof.

In yet another aspect, a method of prophylactic or therapeutic treatment of a human or a veterinary animal is provided, the method comprising designing a dietary and/or a pharmaceutical regimen for the human or veterinary animal based on the human's or animal's cytokine phenotype, i.e., cytokine responsiveness, which regimen is effective at preventing or treating the health conditions diagnosed via the screening methodology and assays. In some embodiments, the prevention/treatment involves administering a flavanol and/or a procyanidin, or any mixture thereof, according to the regimen.

The invention also relates to a method of determining a therapeutic value of a polyphenol not known to have cytokine modulating properties, by testing the polyphenol ex vivo in an assay comprising body samples from at least one low and at least one high cytokine producer and incubating body samples with the polyphenol to determine whether the cytokine levels in the low and high cytokine producer are affected by the presence of the polyphenol.

### DESCRIPTION OF THE DRAWINGS

FIG. 1 represents a scatter plot of all individuals (n=13) tested and their responses to each cocoa flavanol/procyanidin (FLO) fraction. Each open circle represents a value in the form of percentage change (relative to baseline control) from an individual.

FIG. 2 represents the effect of cocoa flavanols and procyanidins (FLO) on secretion of TGF-β1 in low baseline cytokine producers. PBMC were incubated in the presence of individual cocoa fractions (25 μg/ml) for 72 hours before supernatants were extracted for ELISA analysis (mean±SEM; n=7). Values induced from cocoa treatment were compared with control values (i.e., media baseline without cocoa) using a student paired t-test with a two-tailed p-value (* Significance was taken as p<0.05).

FIG. 3 represents the effect of cocoa flavanols and procyanidins (FLO) on secretion of TGF-β1 in high baseline cytokine producers. PBMC were incubated in the presence of individual cocoa fractions (25 μg/ml) for 72 hours before supernatants were extracted for ELISA analysis (mean±SEM; n=7). Values induced from cocoa treatment were compared with control values (i.e., media baseline without cocoa) using a student paired t-test with a two-tailedp-value. (* Significance was taken as p<0.05).

### DETAILED DESCRIPTION OF THE INVENTION

All patents, patent applications and references cited in this application are hereby incorporated herein by reference. In case of any inconsistency, the present disclosure governs.

The invention relates to screening, diagnostic, prophylactic, therapeutic and nutritional applications of flavanols andlor procyanidins, or any mixtures thereof, for use as a screening tool to identify cytokine responsiveness in a human or a veterinary animal, and for diagnosis, prevention andlor treatment of conditions associated with inflammatory andlor immunomodulating pathways. As used herein, an “inflammatory pathway” is a biochemical pathway occurring in the living body of a mammal in response to a harmful stimulus and/or tissue injury. An “immunomodulating pathway” is a biochemical pathway capable of modulating or regulating the immune function in the living body of a mammal. Examples of veterinary animals are cats, dogs and horses.

The invention further relates to design of customized pharmaceutical and/or dietary regimens (i.e., interventions) for humans or veterinary animals to address their health issues identified through the screening methodology described herein. Once the health issue or condition is identified, any approach for preventing or treating the condition may be used. In some embodiments, the regimen may comprise administration of a flavanol and/or a procyanidin, or any mixture thereof.

Compounds and Compositions

The compounds for use in the present invention are flavanols, such as epicatechin, catechin, and gallated forms thereof such as epicatechin gallate and catechin gallate. Procyanidins, which are for purposes of the present applications defined as oligomers of the flavanols may also be used and may contain at least one gallated monomer. Procyanidins include B-type and A-type procyanidins.

Flavanols are monomeric compounds and include (+)-catechin, (−)-epicatechin and their respective epimers (e.g. (−)-catechin and (+)-epicatechin) and have the structure:

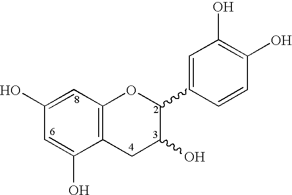

The procyanidin oligomers may have from 2 to about 18, preferably from 2 to about 12, and most preferably from 2 to about 10 monomeric units. At least some of the monomeric units may be gallated. For example, oligomers may be dimers, trimers, tetramers, pentamers, hexamers, septamers, octamers, nonamers and decamers. In a B-type oligomer, monomers shown above are connected via interflavan linkages of (4→6) and/or (4→8). Oligomers with exclusively (4→8) linkages are linear; while the presence of at least one (4→6) bond results in a branched oligomer.

Linear oligomers, wherein n is an integer from 0 to 16 are represented by the following formula:

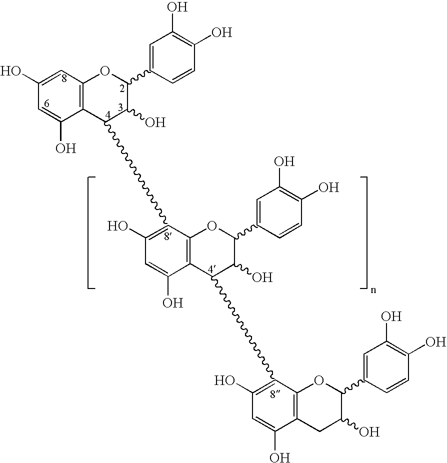

Examples of branched oligomers, wherein A and B are independently oligomers from 1 to 15 which total 3-18 in the final oligomer are represented with the following formula:

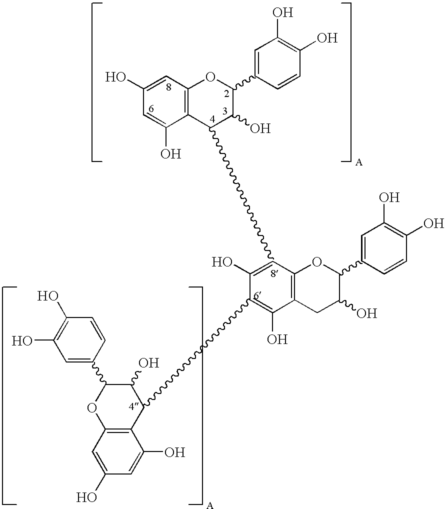

Also useful in the invention are A-type procyanidins, i.e., doubly linked oligomers (comprising the monomers described above) which contain linkages C2-O—C7 and C4→C8 or C4→C6.

The compounds for use in the present invention may be of natural origin or synthetically prepared. Naturally occurring compounds may be isolated from a variety of polyphenol containing compounds, for example from cocoa, grape seeds, peanuts, cranberries, apples and other sources. A person of skill in the art may select natural or synthetic compounds based on availability and/or cost.

Flavanols and procyanidins for use in the present invention that are obtained from cocoa are also referred to herein, for simplicity, as “cocoa polyphenols” (CP). CPs may be derived from cocoa beans, cocoa nibs or cocoa ingredients. The term “cocoa ingredients” refers to a cocoa solids-containing material derived from shell-free cocoa nibs such as chocolate liquor and partially or fully-defatted cocoa solids (e.g. cake or powder). CPs may be included in the compositions of the inventions in the form of a cocoa ingredient, an extract, an extract fraction, or pooled extract fractions.

The cocoa polyphenol may be prepared by extraction from cocoa beans, cocoa nibs, or cocoa ingredients such as chocolate liquor, partially defatted cocoa solids, and/or fully defatted cocoa solids. Preferably, the extract is prepared from a fully or partially defatted cocoa powder. Beans from any species of Theobroma (for example, T. cacao and T grandiflorum), Herrania or inter- and intra-species crosses thereof may be used. The extract may be prepared from fermented, underfermented or unfermented beans, the fermented beans having the least amount of cocoa polyphenols and the unfermented the most. The selection of beans may be made based on the fermentation factor of the beans, for example, the extract may be made from the beans having a fermentation factor of 275 or less. Optimizing the level of polyphenols in the cocoa ingredient and extract thereof by manipulating the degree of fermentation may be done as described in the International Appl. No. PCT/US97/15893 published as WO98/09533, corresponding to U.S. Pat. No. 6,015,913, the relevant portions of which are hereby incorporated herein by reference.

Cocoa polyphenols may be extracted from cocoa ingredients that have been processed using traditional methods of cocoa processing (described, for example, in Industrial Chocolate Manufacture and Use, ed. Beckett, S.T., Blackie Acad. & Professional, New York, 1997, such as in Chapters 1, 5 and 6) or using an improved processing method described in U.S. Pat. No.6,015,913 to Kealey et al. that preserves polyphenols, in contrast to traditional processing methods, by preventing polyphenol destruction. The improved cocoa processing method omits the traditional roasting step. Thus, cocoa ingredients obtainable by (a) heating the cocoa bean for a time and a temperature sufficient to loosen the cocoa shell without roasting the cocoa nib; (b) winnowing the cocoa nib from the cocoa shell; (c) screw pressing the cocoa nib and (d) recovering the cocoa butter and partially defatted cocoa solids which contain preserved levels of cocoa polyphenols, may be used. The method retains a much higher level of higher procyanidin oligomers than traditionally. Cocoa solids produced by this method may contain greater than 20,000 μg of total procyanidins per gram nonfat solids; preferably greater than 25,000 μg/g, more preferably greater than 28,000 μg/g, and most preferably greater than 30,000 μg/g. For purposes of this invention, the total procyanidin amounts are determined as described in Hammerstone et al., (J. Agric. Food Chem., 47:2:490-496, 1999), hereby incorporated herein by reference.

Cocoa polyphenols may be extracted from the sources indicated above using solvents in which the polyphenols dissolve. Suitable solvents include water or organic solvent such as methanol, ethanol, acetone, isopropyl alcohol and ethyl acetate. Solvent mixtures may also be used. When water is used as the solvent, it may be slightly acidified, for example with acetic acid. Preferred solvents are mixtures of water and organic solvent, for example aqueous methanol, ethanol or acetone. Aqueous organic solvents may contain, for example, from about 50% to about 95% of organic solvent. Thus, 50%, 60%, 70%, 80% and 90% organic solvent in water may be used. The solvent may also contain a small amount of acid such as acetic acid, for example, in the amount of about 0.5% to about 1.0%. The composition of the extracts, i.e., the representation (i.e., oligomeric profile) and the amount of procyanidin oligomers, will depend on the choice of solvents. For example, the water extract contains primarily monomers, the ethyl acetate extract contains monomers and lower oligomers, mainly dimers and trimers, and the aqueous methanol, ethanol or acetone extract contains monomers and a range of higher oligomers. One of the preferred solvents for extraction of monomer as well as higher procyanidin oligomers is 70% acetone. However, any extract containing polyphenols is useful in the invention. The methods of cocoa polyphenol extraction are known in the art and are described, for example, in the U.S. Pat. No. 5,554,645 to Romanczyk et al. and the International Appl. No. PCT/US97/05693, published as WO97/36497, both of which are hereby incorporated herein by reference. Thus, in one embodiment, the cocoa extract is prepared by reducing cocoa beans to cocoa powder, defatting the powder, extracting the cocoa polyphenols, and purifying the extract. The cocoa powder can be prepared by freeze-drying the cocoa beans and pulp, depulping and dehulling the freeze-dried cocoa beans, and grinding the dehulled beans.

The cocoa polyphenol extract may be purified, for example, by removal of the caffeine and/or theobromine, and further purified by gel permeation chromatography and/or High Pressure Liquid Chromatography (HPLC). Gel permeation chromatography (e.g. on Sephadex LH-20) may be used, for example, to enrich the extract for higher procyanidin oligomers. For example, the eluate containing monomers and lower oligomers may not be collected until the oligomer(s) of choice begins eluting from the column. An example of such an extract is known in the art and is described in Example 5 of the International Appl. No. PCT/US97/05693, published as WO97/36497, now U.S. Pat. No. 6,297,273, the relevant portions of which are hereby incorporated by reference herein. By using preparative HPLC, for example, normal phase HPLC, the extract may be fractionated, for example, into monomeric and oligomeric fractions containing at least 50% by weight of the monomer or specific oligomer(s). When the fractions contain the monomers and lower oligomers (up to and including the tetramer), the fractions contain about 90 to 95% by weight of the particular oligomeric fraction. The desired fractions may be pooled after separation to obtain a combination of oligomers of choice for example to contain oligomers 3-10 or 5-10. A person of skill in the art can manipulate the chromatographic conditions to achieve the desired procyanidin profile in view of the guidance in this specification, general knowledge in the art and, for example, the teachings of U.S. Pat. No. 5,554,645 to Romanczyk et al. and the International Appl. No. PCT/US97/05693, published as WO97/36497, now U.S. Pat. No. 6,297,273.

Cocoa polyphenols may also be provided in the composition of the invention by cocoa ingredients containing polyphenols or by including chocolate, which may be milk, sweet and semi-sweet, and is preferably dark chocolate, and low fat chocolate. The cocoa ingredients may be prepared using traditional cocoa processing procedures but is preferably prepared using the method described in U.S. Pat. No. 6,015,913 to Kealey et al. Alternatively, to enhance the level of cocoa polyphenols, chocolate liquor and cocoa solids prepared from cocoa beans having a fermentation factor of 275 or less may be used. These ingredients have cocoa polyphenol content that is higher than can be obtained using traditional cocoa processing methods (e.g. with roasting) and fully fermented beans. The chocolate may be prepared using conventional techniques from the ingredients described above or using an improved process for preserving cocoa polyphenols during chocolate manufacturing as described in the International Appl. No. PCT/US99/05414 published as WO99/45788, now US Pat. No. 6,399,139, the relevant portions of which are hereby incorporated herein by reference. A chocolate prepared by at least one of the following non-traditional processes is referred to herein as a “chocolate having a conserved amount of cocoa polyphenols”: (i) preparing cocoa ingredients from underfermented or unfermented cocoa beans; (ii) preserving cocoa polyphenol during cocoa ingredient manufacturing process; and (iii) preserving cocoa polyphenol during chocolate manufacturing process.

Synthetic procyanidins may also be used and are prepared by methods known in the art and as described for example in the International Appl. No. PCT/US98/21392 published as WO99/19319, now U.S. Pat. No. 6,207,842, the relevant portions of which are hereby incorporated herein by reference.

Derivatives of flavanols and procyanidins may also be useful in the present invention. These include gallated monomers and oligomers, glycosylated monomers and oligomers, and mixtures thereof; metabolites of the monomers and oligomers, such as the sulphated, glucuronidated, and methylated forms; and enzyme cleavage products of procyanidins generated by colonic microflora metabolism or internal mammalian metabolism. Examples of derivatives and methods of their making are well known in the art as shown, for example, in U.S. Pat. No. 6,469,053 and International Appl. No. PCT/US00/335331 published as WO01/41775, the relevant portions of each being hereby incorporated herein by reference. The derivatives may be from natural sources or prepared synthetically.

Screening and Diagnostic Assays

Flavanols and procyanidins of the invention may be used as a screening tool to identify cytokine responsiveness in a subject, a human or a veterinary animal, to treatment with flavanols and procyanidins. An important advantage of determining a subject's cytokine responsiveness rests in the ability to design pharmaceutical and/or dietary interventions on an individual basis.

Cytokines include interleukins, lymphokines, chemokines, TNF, interferons and TGF. In one embodiment, the cytokine is TGF beta [herein “TGF-β”], for example TGF beta 1 [herein “TFG-β1”]. Examples of cytokines are IL-1, IL-2, IL-4, IL-S, and TNF-α (alpha). Diagnosis and treatment of diseases or conditions associated with these cytokines are within the scope of the invention.

A screening assay for identifying cytokine responsiveness in a human or a veterinary animal comprises: (i) determining ex vivo a baseline cytokine level in a body sample of a human or a veterinary animal, wherein the cytokine is associated with an inflammatory and/or immunomodulating pathway; and (ii) incubating the body sample with a series of flavanols and procyanidins, or a mixture thereof, under conditions sufficient to induce a change in cytokine levels, and measuring the resulting cytokine levels. The assay may further comprise comparing the baseline cytokine level with the cytokine levels obtained in step (ii) to determine cytokine responsiveness in the human or the veterinary animal to flavanols and procyanidins.

As used herein, a “baseline cytokine level” means the level of a cytokine in the body of a human or a veterinary animal, as determined ex vivo, when such human or animal is not being subjected to any dietary or pharmaceutical treatment designed to modulate, or resulting in modulation of, the cytokine's levels.

Cytokine responsiveness may be identified using an assay described in Example 2. Thus, blood is obtained from a subject, a human or veterinary animal; peripheral blood mononuclear cells (PBMB) are isolated and incubated with individual flavanols and procyanidins. The level of secreted cytokines, for example TGF-β1, is measured in the culture supernatant prior to, and subsequent to, the addition of flavanols and procyanidins. Cytokine responsiveness may be identified using a series of separately tested individual flavanols and procyanidins, for example, monomers, and dimers through decamers, as shown in Example 2, or alternatively, using a mixture of flavanols and procyanidins in a single test sample. A suitable mixture is a cocoa extract comprising flavanols and procyanidin oligomers 2-18, for example monomers and oligomers 2-10. Other assays suitable for measuring the effect of flavanols and procyanidins on cytokine secretion and/or levels in a body sample may also be used.

The results are analyzed to determine the responsiveness of the cytokine to flavanols and procyanidins. For example, a percentage change in cytokine secretion relative to the subject's baseline level is calculated for each tested flavanol and procyanidin, or, in the alternative approach, for a flavanol/procyanidin mixture. The results are of three fold importance.

First, a subject may be identified as a low baseline cytokine producer or a high baseline cytokine producer. This is important because cytokine responsiveness cannot be identified only by observing the subject's baseline cytokine level; an incubation step with flavanols and procyanidins is required. Thus, a subject is identified as a “low baseline producer” if flavanols and procyanidins, or mixtures thereof, increase the baseline cytokine level. In contrast, a subject is identified as a “high baseline producer” if flavanols and procyanidins, or mixtures thereof, decrease the baseline cytokine level. As shown in Example 2, all flavanols and procyanidins possess a cytokine homeostatic, or modulating activity, i.e., they increase or decrease (although to a different degree) cytokine levels depending on the subject's cytokine phenotype. In other words, the same flavanol or procyanidin may either decrease or increase cytokine levels depending on the subject's baseline cytokine levels. For example, as shown for TGF-β1, while some flavanols and procyanidins are more active in one scenario than in the other, the general effect of each compound in each individual was similar in that they stimulated TGF-β1 release from low baseline producers, and inhibited TGF-β1 secretion from high baseline producers.

Second, identifying a subject as a low or high cytokine producer may, depending on the cytokine, be diagnostic of a previously undiagnosed health condition in the subject. It also provides an opportunity for treatment on an individual basis. Thus, a low baseline cytokine producer is likely to be in need of augmenting the cytokine levels, and a high baseline cytokine producer is likely to be in need of reducing the cytokine levels, both with a goal of promoting homeostasis. The compounds disclosed herein may be used to achieve that effect. As will be understood by a person of skill in the art, “homeostasis” refers to a tendency to stability in normal body states of an organism, which is achieved by a system of control mechanisms.

Third, the most effective flavanols and/or procyanidins for treating the subject in need thereof may be identified by selecting the flavanols/procyanidins that exert the most dramatic effect on the baseline cytokine levels. In Example 2, for example, these are monomers and dimers for low baseline producers, and higher oligomers for high baseline producers.

The above described assay may be also used to create a set of standards for any cytokine, which can be tested across a number of subjects (including those that are low, normal and high cytokine level producers) for their response to flavanols and procyanidins. Such standards are helpful for designing a dietary and/or pharmaceutical regimen for a subject depending on that subject's baseline cytokine level—a subject's baseline cytokine level is compared with the standards and appropriate customized regimen, for example a flavanol/procyanidin regimen, is designed. The standards are particularly useful when obtained for a series of cytokines. Depending on the base cytokine level, a dietary and/or pharmaceutical regimen may be designed to modulate the cytokine levels in the subject, i.e., promote the homeostasis of the cytokine levels in the body of a human or a veterinary animal.

As noted above, the above mentioned screening methodology and assays are further useful for early identification (diagnosing) of subjects at risk of conditions associated with an inflammatory and/or an immunomodulatory pathway even when no visible symptoms are present. A person of skill in the art can identify such conditions based on the knowledge in the art relating to the cytokine tested in the screening assay.

For example, TGF-β1 is a multifunctional protein considered to be involved in a variety of physiological processes (Grainger and Metcalfe, in The Endothelium in Clinical Practice, Rubanyi and Dzau, eds (Marcel Dekker Inc, New York) 1997;203-243; Kenny et al., Am Heart J 1994;127:1456-1461). In particular, it has received attention as a potential mediator of cardiovascular protection since Grainger and Metcalfe proposed their protective cytokine hypothesis (Baxter et al., J Cardiovasc Pharm 2001 ;38:930-939, Mao et al., Int J Immunotherapy 1999;15:23-29). This hypothesis is based on the evidence that TGF-β1 actively maintains the normal physiological phenotype of endothelial cells and smooth muscle cells in the arterial vessel wall, thereby inhibiting activation of endothelial cells, as well as suppressing migration, dedifferentiation and proliferation of smooth muscle cells induced by atherogenic agents. In support of TGF-β1 as an inhibitor of atherogenesis, in vivo studies have shown decreased levels of the active form of TGF-β1 in subjects with advanced atherosclerosis (Baxter et al., J Cardiovasc Pharm 2001 ;38:930-939). On the other hand, excess production of TGF-β1 can cause extracellular matrix accumulation that is unfavorable in the injured vessel wall, consequently leading to cardiac fibrosis (Pearson et al., Methods Enzymol 2001;335:350-360). A study exploring the association between TGF-β1 and coronary heart disease (CHD) demonstrated that an increase in the active form of TGF-β1 was associated with the occurrence and severity of CHD (Grainger et al., Hu Mol Gen 1999;8:93-97). Furthermore, another investigation displayed a correlation between a high-producing TGF-β1 genotype and an early onset of coronary vasculopathy following cardiac transplantation (Wang et al., Cardiovasc Res 1997;34:404-410). Finally, TGF-β1 is implicated as a major player in renal disease/failure. Therefore, determining, in a diagnostic assay, an abnormal baseline cytokine level in a subject may aid in early diagnosis and treatment of the subject. Thus, determination of TGF-β1 levels in a subject may lead to early diagnosis and treatment of diseases mentioned above. (e.g. cardiovascular disease, coronary heart disease, cardiac fibrosis, atherosclerosis, renal disease/failure). For example, using the screening methodology of the invention, a human or a veterinary animal (e.g. cat, dog) may be diagnosed as having vascular and renal health issues such as being at risk of, or suffering from, cardiovascular disease, coronary heart disease, cardiac fibrosis, atherosclerosis, and/or renal disease or failure.

Similarly, IL-4 levels are relevant for the treatment of allergy, rheumatoid arthritis (Gallagher et al., Curr. Opin. Rheumatol., 11(5):372-6, 1999), asthma (Pauwels et al., Clin. Exp. Allergy, 28 Suppl. 3:1-5, 1998), and inflammatory immune disease (Rocken et al., Immunol Today 17(5):225-31, 1996). IL-4 may also have a role in preventing the onset of insulin-dependent diabetes (Cameron et al., Crit Rev Immunol 17(5-6):537-44, 1997). Down-regulation of IL-6 has been used in the treatment of inflammatory bowel disease (Rogler et al., World J Surg 22(4):382-9, 1998). IL-6 plays a significant role in the induction of inflammatory skin diseases suggesting down-regulation of IL-6 production as a therapy for this condition (Sawamura et al., J Immunol, 161 (10):5633-9, 1998). IL-5 has been implicated as an important player in inflammation and conditions such as asthma and periodontal disease, and TNFα has been implicated with inflammatory bowel disease (Sandbom et al., Inflamm. Bowel Dis. 5(2), 119-33, 1999) and rheumatoid arthritis (Ohshima et al., J Clin Immunol, 19(5):305-13, 1999). Conditions associated with these and other cytokines may be diagnosed early using the screening methodology of the invention.

Design of Dietary and Pharmaceutical Regimens

A dietary and/or a pharmaceutical regimen or intervention for a particular human or a veterinary animal can now be custom designed based on the human's or animal's cytokine phenotype, i.e., cytokine production, and the methods for prophylactic or therapeutic treatment can be advantageously optimized. The prophylactic or therapeutic treatment may be achieved using, for example, administration of flavanols and/or procyanidins, however, any other prophylaxis or therapy known to treat conditions associated with inflammatory and/or immunomodulating pathways may be used.

The method of designing a dietary and/or a pharmaceutical regimen generally comprises: (i) determining ex vivo a baseline cytokine level in a body sample of the human or the veterinary animal, wherein the cytokine is associated with an inflammatory and/or immunomodulating pathway; and (ii) designing, based on the baseline cytokine level, a pharmaceutical and/or dietary regimen for (depending on the subject's condition) maintenance of the subject's healthy condition or for prevention and/or treatment of conditions associated with inflammatory and/or immunomodulating pathways. In one embodiment, the cytokine is TGFβ, more specifically TGF-β1.

The custom designed regimen may maintain, increase, or decrease the baseline cytokine levels in the human or the veterinary animal or otherwise prevent the progression of, or treat, a health condition associated with an inflammatory and/or immunomodulating pathway. In some embodiments, the pharmaceutical and/or dietary regimen will contemplate administration of a flavanol and/or a procyanidin oligomer. However, any other regimen known to prevent, prevent the progression of, and/or treat conditions associated with inflammatory and/or immunomodulating pathways may be used. Even treatments well known in the art can advantageously and unexpectedly benefit from the determination of the subject's cytokine phenotype and responsiveness.

The method may further comprise a screening assay for identifying the cytokine responsiveness (e.g. TGFβ, more specifically TGFβ1 responsiveness) to flavanols and procyanidins. The assay may be performed as described in the preceding section and in Example 2.

In one embodiment, a method is provided for designing a dietary and/or a pharmaceutical regimen for a human or a veterinary animal comprising: (i) determining ex vivo a baseline cytokine level in a body sample of the human or the veterinary animal, wherein the cytokine is associated with an inflammatory and/or immunomodulating pathway; (ii) diagnosing, based on the baseline cytokine level, whether the human or the veterinary animal is at risk of, or suffers from, a condition associated with inflammatory and/or immunomodulating pathways; and (iii) designing a pharmaceutical and/or dietary regimen effective for prophylactic or therapeutic treatment of the condition diagnosed in step (ii). The regimen may comprise administration of flavanols, procyanidins or mixture thereof, but other approaches, which will be apparent to those of skill in the art may also be used.

In another embodiment, the invention provides for a method of designing a dietary and/or a pharmaceutical regimen for a human or a veterinary animal comprising: (i) determining ex vivo a baseline cytokine level in a body sample of the human or the veterinary animal, wherein the cytokine is associated with an inflammatory and/or immunomodulating pathway; (ii) incubating the body sample with a series of flavanols and procyanidins, or a mixture thereof, under conditions sufficient to induce a change in cytokine levels and measuring the resulting cytokine levels; (iii) comparing the baseline cytokine level with the cytokine levels obtained in step (ii) to determine cytokine responsiveness of the human or the veterinary animal to flavanols and procyanidins; and (iv) designing, based on the cytokine responsiveness, a pharmaceutical and/or dietary regimen to promote homeostatic cytokine levels in the human or the veterinary animal; wherein the pharmaceutical and/or dietary regimen comprises administration of a flavanol and/or a procyanidin oligomer, or a mixture thereof.

Also within the scope of the invention is a method of designing a dietary and/or a pharmaceutical regimen for a human or a veterinary animal comprising: (i) determining ex vivo a baseline cytokine level in a body sample of the human or the veterinary animal, wherein the cytokine is associated with an inflammatory and/or immunomodulating pathway; (ii) incubating the body sample with a series of flavanols and procyanidins, or a mixture thereof, under conditions sufficient to induce a change in cytokine levels and measuring the resulting cytokine levels; (iii) comparing the baseline cytokine level with the cytokine levels obtained in step (ii) to determine cytokine responsiveness of the human or the veterinary animal to flavanols and procyanidins; (iv) diagnosing, based on the cytokine responsiveness, whether the human or the veterinary animal is at risk of, or suffers from, a condition associated with inflammatory and/or immunomodulating pathways; and (v) designing a pharmaceutical and/or dietary regimen effective for prophylactic or therapeutic treatment of the condition diagnosed in step (iv). The pharmaceutical and/or dietary regimen may comprise administration of a flavanol and/or a procyanidin oligomer but other suitable regimens may be used. Examples of conditions that may be diagnosed are cardiovascular disease, coronary heart disease, cardiac fibrosis, atherosclerosis, and/or renal disease or failure.

Depending on whether a human or a veterinary animal is in need of increasing, decreasing or maintaining the cytokine levels, a regimen comprising administration of selected flavanols and/or procyanidins, or mixtures thereof, is designed. The present invention is also useful for the subjects that have normal baseline cytokine levels since a mixture of the compounds described herein may be administered for prophylaxis to maintain the homeostatic cytokine levels. For example, a dietary or pharmaceutical regimen may be designed and involve administration of a mixture of lower and higher procyanidin oligomers which will maintain the baseline cytokine levels.

Treatment Methods

Methods of prophylactic or therapeutic treatment of a human or a veterinary animal is also within the scope of the present invention. The method comprises designing a dietary and/or a pharmaceutical regimen for the human or veterinary animal based on the human's or animal's cytokine phenotype as described above, and administering a flavanol and/or a procyanidin according to the regimen.

In one embodiment, the method provides for prophylactic or therapeutic treatment of a human or a veterinary animal comprising: (i) determining ex vivo a baseline cytokine level in a body sample of the human or the veterinary animal, wherein the cytokine is associated with an inflammatory and/or immunomodulating pathway; (ii) incubating the body sample with a series of flavanols and procyanidins, or a mixture thereof, under conditions sufficient to induce a change in cytokine levels, and measuring the resulting cytokine levels; (iii) comparing the baseline cytokine level with the cytokine levels obtained in step (ii) to determine cytokine responsiveness of the human or the veterinary animal to flavanols and procyanidins; (iv) designing, based on the cytokine responsiveness, a pharmaceutical and/or dietary regimen to promote homeostatic cytokine levels in the human or the veterinary animal; wherein the pharmaceutical and/or dietary regimen comprises administration of a flavanol and/or a procyanidin oligomer; and (v) administering a flavanol and/or a procyanidin oligomer, or a mixture thereof, to the human or the veterinary animal according to the pharmaceutical and/or dietary regimen designed in step (iv).

Also provided are methods of prophylactic or therapeutic treatment of a human or a veterinary animal comprising: (i) determining ex vivo a baseline cytokine level in a body sample of the human or the veterinary animal, wherein the cytokine is associated with an inflammatory and/or immunomodulating pathway; (ii) diagnosing, based on the baseline cytokine level, whether the human or the veterinary animal is at risk of, or suffers from, a condition associated with inflammatory and/or immunomodulating pathways; (iii) designing a pharmaceutical and/or dietary regimen effective for prophylactic or therapeutic treatment of the condition diagnosed in step (ii); and (iv) treating the human or the veterinary animal according to the pharmaceutical and/or dietary regimen designed in step (iii). In some embodiments, the pharmaceutical and/or dietary regimen comprises administration of a flavanol and/or a procyanidin oligomer but other approaches may be used. Examples of the diagnosed condition are cardiovascular disease, coronary heart disease, cardiac fibrosis, atherosclerosis, and/or renal disease or failure.

Another embodiment involves a method of prophylactic or therapeutic treatment of a human or a veterinary animal comprising: (i) determining ex vivo a baseline cytokine level in a body sample of the human or the veterinary animal, wherein the cytokine is associated with an inflammatory and/or immunomodulating pathway; (ii) incubating the body sample with a series of flavanols and procyanidins, or a mixture thereof, under conditions sufficient to induce a change in cytokine levels, and measuring the resulting cytokine levels; (iii) comparing the baseline cytokine level with the cytokine levels obtained in step (ii) to determine cytokine responsiveness of the human or the veterinary animal to flavanols and procyanidins; (iv) diagnosing, based on the baseline cytokine level, whether the human or the veterinary animal is at risk of, or suffers from, a condition associated with inflammatory and/or immunomodulating pathways; (v) designing a pharmaceutical and/or dietary regimen effective for prophylactic or therapeutic treatment of the condition diagnosed in step (iv); and (vi) treating the human or the veterinary animal according to the pharmaceutical and/or dietary regimen designed in step (v). The pharmaceutical and/or dietary regimen may comprise administration of a flavanol and/or a procyanidin oligomer but other approaches apparent to those of skill in the art may be used. The conditions to be treated may be, inter alia, cardiovascular disease, coronary heart disease, cardiac fibrosis, atherosclerosis, and/or renal disease or failure.

In more specific embodiments, methods for treating low baseline TGF-β1 producers and high baseline TGF-β1 producers are contemplated. Thus, two following methods are also within the scope of the invention: (i) a method of treating a subject, which is a low baseline TGF-β1 producer, comprising administering to the subject at least one flavanol and/or procyanidin oligomer selected from the group consisting of monomer, dimer, trimer, tetramer and pentamer, or any mixture thereof, in the amount effective to stimulate the level of TGF-β in the subject, wherein the subject is a human or a veterinary animal; and (ii) a method of treating a subject, which is a high baseline TGF-β producer, comprising administering to the subject at least one procyanidin oligomer 6-10, or any mixture thereof, in the amount effective to stimulate the level of TGF-β in the subject, wherein the subject is a human or a veterinary animal.

Patients at risk of, or suffering from, conditions associated with an inflammatory and/or immunomodulating pathway may be phenotyped, diagnosed and/or treated as described herein. Examples of such conditions are cardiovascular disease, coronary heart disease, atherosclerosis, cardiac fibrosis, thrombosis (e.g. deep vein thrombosis) and other vascular conditions generally, as well as asthma, inflammatory bowel disease, ulcerative colitis, Chron's disease, gingivitis, periodontitis, acute edema, and arthritis (e.g. rheumatoid arthritis).

Flavanols, procyanidins and derivatives thereof are administered in the effective amount. A person of skill in the art can determine the effective amounts using the general knowledge in the art and the guidance in this application. For example, the compounds of the invention may be administered in the amount of at least about 50 mg per day to about several grams a day. The high end amounts are not limited. In one embodiment, the effective amounts are from about 100 mg to about 2 grams, or from about 100 mg to about 1.5 g, or from about 200 to about 600 mg. The compounds can be administered once or several times (e.g. 2 or 3) a day taking into consideration the half life of the compounds in the body of a subject. The compounds may be administered in a regimen designed by a person of skill in the art, and may be daily, weekly, monthly etc.

Flavanols and/or procyanidins may be administered in the form of a food, a food additive, a dietary supplement, or a pharmaceutical. Such compositions may contain a carrier, a diluent, or an excepient. Depending on the intended use, the carrier, diluent, or excepient may be chosen to be suitable for human or veterinary use, food, additive, supplement or pharmaceutical use.

As used herein a “food” is a material consisting essentially of protein, carbohydrate and/or fat, which is used in the body of an organism to sustain growth, repair and vital processes and to furnish energy. Foods may also contain supplementary substances such as minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R. 170.3(e)(1) and includes direct and indirect additives. As used herein, a “pharmaceutical” is a medicinal drug. See Merriam-Webster's Collegiate Dictionary, 10th Edition, 1993. A pharmaceutical may also be referred to as a medicament. As used herein, a “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients.

Any conventional food including any beverage which has been improved with by augmenting the levels of flavanols/procyanidins is within the scope of the invention. In the case of cocoa polyphenol, the improvement is achieved either (i) by adding cocoa polyphenol or a derivative thereof to a food that does not contain cocoa polyphenol or (ii) when the food traditionally contains cocoa polyphenols, such as for example chocolate, by enhancing the polyphenol level over the one found in the traditionally prepared food. The enhancement may be achieved by adding additional cocoa polyphenols, for example, in a form of an extract; by adding cocoa polyphenol in combination with another polyphenol containing ingredient (e.g. nut skins); by manipulating the cocoa ingredients processing and cocoa bean selection, as described above, to preserve cocoa polyphenol in the cocoa ingredient used for the manufacture of the food product; or by manipulating the chocolate manufacturing process as described above. Thus, these foods (including beverages) contain an “elevated level of polyphenols” (including cocoa procyanidins) in comparison to comparative conventional foods (including beverages). An example of a chocolate having an elevated level of polyphenol occurs when a chocolate manufacturer adds a cocoa extract containing cocoa polyphenols to its previously commercially available product. The foods may also be referred to as “high cocoa polyphenol foods,” i.e., they contain higher levels of polyphenol than their traditional counterparts.

In one embodiment the food is a confectionery such as a standard of identity (SOI) and non-SOI chocolate, such as milk, sweet and semi-sweet chocolate including dark chocolate, low fat chocolate and a candy which may be a chocolate covered candy. Other examples include a baked product (e.g. brownie, baked snack, cookie, biscuit) a condiment, a granola bar, a toffee chew, a meal replacement bar, a spread, a syrup, a powder beverage mix, a cocoa or a chocolate flavored beverage, a pudding, a rice cake, a rice mix, a savory sauce and the like. If desired, the foods may be chocolate or cocoa flavored. Food products may also contain L-arginine and/or a cholesterol lowering agent.

The compositions may be administered to a healthy mammal for prophylactic purposes or to a mammal in need of a treatment or having at least one of the risk factors associated. Any individual having at least one of the risk factors associated with vascular health problems is a subject for administration of the compositions described herein. The individuals with a familial history of elevated cholesterol levels, peri- or post-menopausal females, postmenoposal females w/myocardial post-ischaemic damage, surgically or chemically induced estrogen deficient females, the aged, those with hyperglycemia, diabetes, hypertension, and obesity, and cigarette smokers are all susceptible individuals in need of the treatment described herein. Other populations of mammals that are susceptible to developing vascular health problems or that have been identified as at risk of developing a condition associated with inflammatory and/or immunomodulating pathways using the assays described herein may also received the composition according to the designed regimen.

Screening of other Polyphenols

The invention also relates to a method designed to evaluate whether other polyphenols have the ability to promote homeostasis.

The method of determining a therapeutic value of a polyphenol to modulate cytokine levels in a mammal depending on the mammal's cytokine production is performed by testing the polyphenol ex vivo in an assay comprising body samples from at least one low and at least one high cytokine respondent (as determined using flavanols and procyanidins in the screening assays described herein) and comparing the effect of the polyphenol on the low cytokine respondent with its effect on the high cytokine respondent to determine which mammalian cytokine phenotype, if any, is affected by the presence of the polyphenol.

In one embodiment, the invention relates to a method of determining a therapeutic value of a polyphenol for modulating cytokine levels in a mammal, the method comprising: (i) obtaining body samples from at least one low cytokine producer and at least one high cytokine producer; (ii) determining baseline cytokine levels in the body samples; (iii) incubating the body samples with a polyphenol not known to have cytokine modulation properties under conditions sufficient to induce a change in cytokine levels; (iv) determining the cytokine levels after the incubation of step (iii); and (v) comparing the baseline cytokine levels with the cytokine levels of step (iv) to determine whether the polyphenol has cytokine modulating properties.

The invention is further described in the following non-limiting examples.

### EXAMPLES

### Example 1

### Extraction and Purification

Procvanidin Extraction Procedures

Method 1

Procyanidins were extracted from the defatted, unfermented, freeze dried cocoa beans using a modification of the method described by Jalal and Collin (‘Polyphenols of Mature Plant, Seedling and Tissue Cultures of Theobroma Cacoa, Phytochemistry, 6, 1377-1380, 1977). Procyanidins were extracted from 50 gram batches of the defatted cocoa mass with 2× 400 mL 70% acetone/deionized water followed by 400 mL 70% methanol/deionized water. The extracts were pooled and the solvents removed by evaporation at 45° C. with a rotary evaporator held under partial vacuum. The resultant aqueous phase was diluted to 1 L with deionized water and extracted 2× with 400 mL CHCl₃. The solvent phase was discarded. The aqueous phase was then extracted 4× with 500 mL ethyl acetate. Any resultant emulsions were broken by centrifugation on a Sorvall RC 28S centrifuge operated at 2,000× for 30 min. at 10° C. To the combined ethyl acetate extracts, 100-200 mL deionized water was added. The solvent was removed by evaporation at 45° C. with a rotary evaporator held under partial vacuum. The resultant aqueous phase was frozen in liquid N₂ followed by freeze drying on a LABCONCO Freeze Dry System. The yields of crude procyanidins that were obtained from the different cocoa genotypes are listed in Table 1.

TABLE 1
Crude Procyanidin Yields

| GENOTYPE | ORIGIN | HORTICULTUREAL RACE |
|---|---|---|
| UIT-1 | Malaysia | 3.81 |
| Unknown | West Africa | 2.55 |
| ICS-100 | Brazil | 3.42 |
| ICS-39 | Brazil | 3.45 |
| UF-613 | Brazil | 2.98 |
| EEG-48 | Brazil | 3.15 |
| UF-12 | Brazil | 1.21 |
| NA-33 | Brazil | 2.23 |

Method 2

Alternatively, procyanidins are extracted from defatted, unfermented, freeze dried cocoa beans with 70% aqueous acetone. Ten grams of defatted material was slurried with 100 mL solvent for 5-10 min. The slurry was centrifuged for 15 min. at 4° C. at 3000×g and the supernatant passed through glass wool. The filtrate was subjected to distillation under partial vacuum and the resultant aqueous phase frozen in liquid N₂, followed by freeze drying on a LABCONCO Freeze Dry System. The yields of crude procyanidins ranged from 15-20%.

Without wishing to be bound by any particular theory, it is believed that the differences in crude yields reflected variations encountered with different genotypes, geographical origin, horticultural race, and method of preparation.

Partial Purification of Cocoa Procyanidins by Gel Permeation Chromatography

Method 1

Procyanidins obtained as described above were partially purified by liquid chromatography on Sephadex LH-20 (28×2.5 cm). Separations were aided by a step gradient from deionized water into methanol. The initial gradient composition started with 15% methanol in deionized water which was followed step wise every 30 min. with 25% methanol in deionized water, 35% methanol in deionized water, 70% methanol in deionized water, and finally 100% methanol. The effluent following the elution of the xanthine alkaloids (caffeine and theobromine) was collected as a single fraction. The fraction yielded a xanthine alkaloid free subfraction which was submitted to further subfractionation to yield five subfractions designated MM2A through MM2E. The solvent was removed from each subfraction by evaporation at 45° C. with a rotary evaporator held under partial vacuum. The resultant aqueous phase was frozen in liquid N₂ and freeze dried overnight on a LABCONCO Freeze Dry System. Approximately, 100 mg of material was subfractionated in this manner.

Chromatographic Conditions: Column; 28×2.5 cm Sephadex LH-20, Mobile Phase: Methanol/Water Step Gradient, 15:85, 25:75, 35:65, 70:30, 100:0 Stepped at ½ Hour Intervals, Flow Rate; 1.5 mL/min, Detector; UV at lambda₁ (λ₁)=254 nm and λ₂=365 nm, Chart Speed: 0.5 mm/min, Column Load; 120 mg.

Method 2

Procyanidins obtained as described above were partially purified by liquid chromatography on Sephadex LH 20 (72.5×2.5 cm), using 100% methanol as the eluting solvent, at a flow rate of 3.5 mL/min. Fractions of the eluent were collected after the first 1.5 hours, and the fractions were concentrated by a rotary evaporator, redissolved in water and freeze dried. These fractions were referred to as pentamer enriched fractions.

The flavanols and procyanidins may be separated in individual fractions using HPLC as described, for example in, U.S. Pat. Nos. 5,554,645 and 6,297,273, the relevant portions of which are hereby incorporated herein by reference.

### Example 2

### Effect of Flavanols and Procyanidins on TGF-β₁

Cocoa Fraction Preparation

Water soluble flavanol and/or procyanidin (FP) fractions were prepared from a high procyanidin content cocoa powder (Cocoapro™, Mars, Incorporated; Hackettstown, N.J.). The powder was prepared according to the method described in the U.S. Pat No. 6,015,913 to Kealey et al., hereby incorporated herein by reference. The cocoa powder was extracted with acetone/water as described in Example 1 to obtain a crude extract. The fractions were purified from the crude extract using high performance liquid chromatography (HPLC) according to Adamson et al (J Agric Food Chem 1999;47:4184-4188.). Purified fractions of monomer through decamers were investigated. The purified FP fractions contained less than 0.5% (w/w) of total alkaloids (theobromine and caffeine). The monomer and procyanidin composition, estimated by HPLC, and molecular weights of these preparations are shown in Table 2. All samples were suspended in RPMI 1640 (Gibco BRL, Gaithersburg, Md.) with 10% heat inactivated fetal bovine serum (Atlanta Biologicals, Norcross, Ga.). They were then diluted with the same medium to final concentrations of 25 μg/ml.

PBMC Isolation

Peripheral blood from healthy volunteers was collected into sodium citrate-containing tubes and mixed 1:1 with Hanks' Balanced Salt Solution (HBSS; Gibco BRL) without calcium chloride, magnesium chloride, or magnesium sulfate. The diluted blood was then layered over a Histopaque®-1077 gradient (Sigma, St. Louis, Mo.) and centrifuged at 500×g for 30 min at room temperature. PBMC were harvested from the interface layer, washed twice with HBSS, and then counted. The cells were resuspended in RPMI 1640 containing 10% fetal bovine serum and supplemented with 0.1% of a 50 mg/ml gentamicin solution (Gibco BRL). PBMC concentration was adjusted to 2×10⁶ viable cells/ml after estimation of viability by trypan blue exclusion assay. Viability was consistently greater that 96%.

Culture of PBMC with Cocoa FP Fractions

Five hundred μl of a 1.0×10⁶ cell suspension were cultured with equal volumes of the various cocoa treatments at 37° C. with 5% CO₂ in 48-well plates. Resting PBMC were incubated with individual cocoa FP fractions at 25 μg/ml. All treatments were performed in duplicate. Following 72 h incubation, the supernatant fractions were harvested for ELISA analysis.

TGF-β1 (ELISA)

Culture supernatant fractions were harvested after 72 h and stored at −20° C. until analysis by ELISA. Ninety-six well Costar EIA plates (Cat. #2592) were coated with mouse anti-TGF-β1 supplied in the DuoSet Human TGF-β1 ELISA Development Kit (R&D Systems, Minneapolis, Minn.). Cell culture supemates containing the latent form of TGF-β1 were activated in an acidic environment (0.5 ml sample +0.1 ml 1N HCl) and neutralized with 0.1 ml of 1.2N NaOH/0.5M HEPES. Subsequently, the activated supemates were measured for TGF-β1 concentrations according to the manufacturers' recommendations. The lowest TGF-β1 standard for the ELISA system was 31.3 pg/ml.

Statistics

The effects of different cocoa FP fractions on the secretion of TGF-β1 were examined in unstimulated resting PBMC. Results were compared by Student paired t-test with a two-tailed p-value (i.e., control cells without cocoa flavonoids versus cells treated with individual FP fractions). Significance was taken as p<0.05.

Results

Unstimulated resting PBMC were prepared and incubated with individual cocoa FP fractions at 25 μg/ml. TGF-β1 production was assessed in the supernatant fractions after 72 h of incubation.

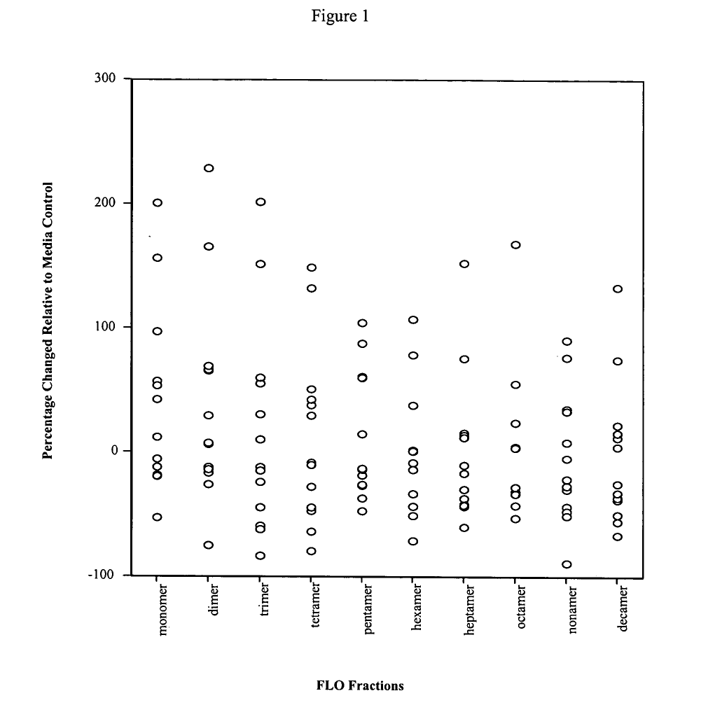

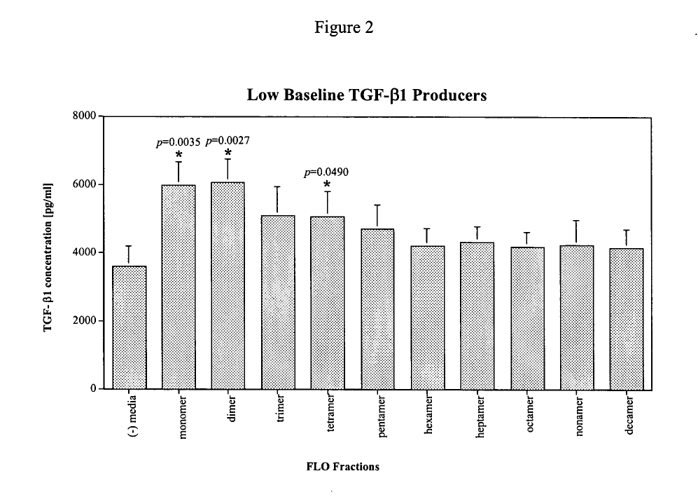

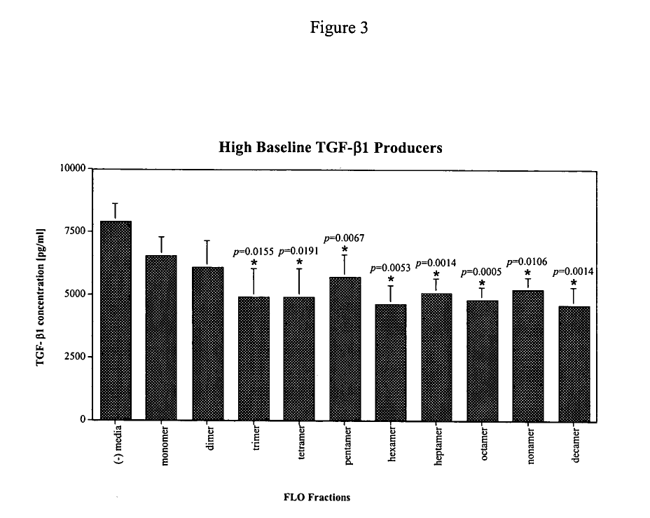

ELISA analysis showed that inter-individual variability was high among the thirteen subjects tested. FIG. 1 depicts the fluctuating response of these individuals to cocoa FP fractions in the form of percentage change relative to the media baseline for each subject. However, when individuals were categorized based on their baseline production of TGF-β1, clear trends, could be observed in the way TGF-β1 secretion was influenced by cocoa FP fractions. There were seven low baseline producers (LBP) whose baseline TGF-β1 concentrations were less than 6000 pg/ml (3604±568 pg /ml), while the rest were assigned to a high baseline producing group (HBP; 7910±695 pg/ml). Individual cocoa FP fractions were stimulatory for TGF-β1 release in the low LBP group (FIG. 2). In general, low molecular weight FP fractions (≦pentamer) were more effective than the larger oligomers in augmentation, inducing increases ranging from 30% to 68% over baseline (Table 3), while the larger oligomers (≦hexamer) only moderately increased TGF-β1 secretion relative to baseline (15% to 20%; Table 3). The monomeric and dimeric FP fractions markedly enhanced TGF-β1 secretion in the LBP group, producing concentrations of 5981±666 (p=0.0035) and 6062±667 (p=0.0027) pg/ml, respectively. In contrast to the LBP group, individual cocoa FP fractions were inhibitory for TGF-β₁ secretion in HBP (FIG. 3). The trimeric through decameric FP fractions significantly suppressed TGF-β1 levels by 28% to 42% relative to baseline (Table 3), while the monomer and dimer showed moderate reductions (17% and 23%, respectively).

The results establish that cocoa FP fractions are able to promote homeostatic levels of TGF-β1 by either augmenting, or suppressing, TGF-β1 release depending on an individual's baseline level of TGF-β1.

In the present study, an evaluation of baseline secretions of TGF-β1 showed a large inter-individual variability among the subjects examined. Grainger et al. have shown that the circulating concentration of TGF-β1 can vary considerably based on the genetic background of the individual (Hu Mol Gen 1999;8:93-97). It is understandable that such disparate baseline levels of TGF-β1 are observed here given that polymorphisms in the TGF-β1 gene can influence its production. Unfortunately, in the current study the genotypic analysis on the subjects tested was not perfomred. Nevertheless, it is clear that, cocoa FP fractions were stimulatory for TGF-β1 protein secretion in PBMC from subjects whose baseline levels of TGF-β1 were low (3604±568 pg/ml). In contrast to low baseline subjects, PBMC from high baseline producing individuals (7910±695 pg/ml) showed suppressed TGF-β1 production following incubation with FP fractions. Since there was no genotype analysis of low and high TGF-β1 producing individuals, it is also possible that HBP were primed to produce TGF-β1 prior to collecting blood from these subjects. Nevertheless, cocoa FP fractions effectively reduced, or enhanced, TGF-β1 secretion in HBP and LBP, respectively.

The effects of cocoa FP on cytokine production, a biphasic type effect was observed previously with the larger and smaller procyanidin fractions showing differential effects on cytokine production. In resting PBMC, the larger FP oligomers (hexamers and above) markedly stimulated IL-β1 and IL-4 release, while the smaller fractions inhibited their secretion (Mao et al., Life Sciences 2000;66:1377-1386; Mao et al., J Medicinal Foods 2000;3:107-114). However, in the present investigation, it was surprisingly discovered that the effect of FP on TGF-β1 release was dependent not only on the molecular size of the FP fractions, but also by the capacity of the PBMC to secrete TGF-β1. Some fractions were more active, with the general effect of cocoa fractions in each individual being similar in that they stimulated TGF-β1 release from LBP, and inhibited TGF-β1 secretion from HBP. Given the above, cocoa FP, in concert with their effects on platelet reactivity, eicosanoid production, and vascular reactivity, also have protective effects on the cardiovascular system by promoting the maintenance of homeostatic TGF-β1 levels.

TABLE 2
Profile of individual cocoa FLO fractions.

| Fraction | Molecular | Procyanidin | |
|---|---|---|---|
| Name | Weight (Da) | Profile | % |
| Monomer | 290 | Monomer | 95 |
| Dimer | 578 | Dimer | 98 |
| Trimer | 866 | Trimer | 93 |
| Tetramer | 1154 | Tetramer | 93 |
| Pentamer | 1442 | Pentamer | 93 |
| Hexamer | 1730 | Hexamer | 89 |
| Heptamer | 2018 | Heptamer | 79 |
| | | Hexamer | 18 |
| Octamer | 2306 | Octamer | 76 |
| | | Heptamer | 16 |
| Nonamer | 2594 | Nonamer | 60 |
| | | Octamer | 28 |
| Decamer | 2882 | Decamer | 40 |
| | | Nonamer | 17 |
| | | Octamer | 22 |
| | | Heptamer | 16 |

TABLE 3
Effect of cocoa FLO fractions on TGF-β secretion in low
(n = 7) and high (n = 6) baseline producers.
Values are expressed as mean percent change from
media baseline control.

| FLO Fraction | Low Baseline Producers | High Baseline Producers |
|---|---|---|
| Monomer | +66% | −17% |
| Dimer | +68% | −23% |
| Trimer | +42% | −38% |
| Tetramer | +40% | −38% |
| Pentamer | +30% | −28% |
| Hexamer | +17% | −41% |
| Heptamer | +20% | −36% |
| Octamer | +16% | −39% |
| Nonamer | +17% | −34% |
| Decamer | +15% | −42% |

What is claimed is:

1. A method of designing a dietary and/or a pharmaceutical regimen for a human or a veterinary animal comprising
(i) determining ex vivo a baseline cytokine level in a body sample of the human or the veterinary animal, wherein the cytokine is associated with an inflammatory and/or immunomodulating pathway; and
(ii) designing, based on the baseline cytokine level, a pharmaceutical and/or dietary regimen to promote homeostatic cytokine levels in the human or the veterinary animal, wherein the pharmaceutical and/or dietary regimen comprises administration of a flavanol and/or a procyanidin oligomer and/or a derivative thereof.

2. A method of designing a dietary and/or a pharmaceutical regimen for a human or a veterinary animal comprising:
(i) determining ex vivo a baseline cytokine level in a body sample of the human or the veterinary animal, wherein the cytokine is associated with an inflammatory and/or immunomodulating pathway;
(ii) diagnosing, based on the baseline cytokine level, whether the human or the veterinary animal is at risk of, or suffers from, a condition associated with inflammatory and/or immunomodulating pathways; and
(iii) designing a pharmaceutical and/or dietary regimen effective for prophylactic or therapeutic treatment of the condition diagnosed in step (ii).

3. The method of claim 2, wherein the pharmaceutical and/or dietary regimen comprises administration of a flavanol and/or a procyanidin oligomer and/or a derivative thereof.

4. The method of claim 2, wherein the condition is cardiovascular disease, coronary heart disease, cardiac fibrosis, atherosclerosis, and/or renal disease or failure.

5. A method of designing a dietary and/or a pharmaceutical regimen for a human or a veterinary animal comprising
(i) determining ex vivo a baseline cytokine level in a body sample of the human or the veterinary animal, wherein the cytokine is associated with an inflammatory and/or immunomodulating pathway;
(ii) incubating the body sample with a series of flavanols and procyanidins, and/or derivatives thereof, or a mixture thereof, under conditions sufficient to induce a change in cytokine levels and measuring the resulting cytokine levels;
(iii) comparing the baseline cytokine level with the cytokine levels obtained in step (ii) to determine cytokine responsiveness of the human or the veterinary animal to flavanols and procyanidins;
(iv) designing, based on the cytokine responsiveness, a pharmaceutical and/or dietary regimen to promote homeostatic cytokine levels in the human or the veterinary animal, wherein the pharmaceutical and/or dietary regimen comprises administration of a flavanol and/or a procyanidin oligomer and/or a derivative thereof, or a mixture thereof.

6. A method of designing a dietary and/or a pharmaceutical regimen for a human or a veterinary animal comprising
(i) determining ex vivo a baseline cytokine level in a body sample of the human or the veterinary animal, wherein the cytokine is associated with an inflammatory and/or immunomodulating pathway;
(ii) incubating the body sample with a series of flavanols and procyanidins and/or derivatives thereof, or a mixture thereof, under conditions sufficient to induce a change in cytokine levels and measuring the resulting cytokine levels;
(iii) comparing the baseline cytokine level with the cytokine levels obtained in step (ii) to determine cytokine responsiveness of the human or the veterinary animal to flavanols and procyanidins;
(iv) diagnosing, based on the cytokine responsiveness, whether the human or the veterinary animal is at risk of, or suffers from, a condition associated with inflammatory and/or immunomodulating pathways; and
(v) designing a pharmaceutical and/or dietary regimen effective for prophylactic or therapeutic treatment of the condition diagnosed in step (iv).

7. The method of claim 6, wherein the pharmaceutical and/or dietary regimen comprises administration of a flavanol and/or a procyanidin oligomer and/or a derivative thereof.

8. The method of claim 6, wherein the condition is cardiovascular disease, coronary heart disease, cardiac fibrosis, atherosclerosis, and/or renal disease or failure.

9. A method of prophylactic or therapeutic treatment of a human or a veterinary animal comprising:
(i) determining ex vivo a baseline cytokine level in a body sample of the human or the veterinary animal, wherein the cytokine is associated with an inflammatory and/or immunomodulating pathway;
(ii) designing, based on the baseline cytokine level, a pharmaceutical and/or dietary regimen to promote homeostatic cytokine levels in the human or the veterinary animal; wherein the pharmaceutical and/or dietary regimen comprises administration of a flavanol and/or a procyanidin oligomer; and
(iii) administering a flavanol and/or a procyanidin oligomer and/or a derivative thereof to the human or the veterinary animal according to the pharmaceutical and/or dietary regimen designed in step (ii).

10. A method of prophylactic or therapeutic treatment of a human or a veterinary animal comprising:
(i) determining ex vivo a baseline cytokine level in a body sample of the human or the veterinary animal, wherein the cytokine is associated with an inflammatory and/or immunomodulating pathway;
(ii) diagnosing, based on the baseline cytokine level, whether the human or the veterinary animal is at risk of, or suffers from, a condition associated with inflammatory and/or immunomodulating pathways;
(iii) designing a pharmaceutical and/or dietary regimen effective for prophylactic or therapeutic treatment of the condition diagnosed in step (ii); and
(iv) treating the human or the veterinary animal according to the pharmaceutical and/or dietary regimen designed in step (iii).

11. The method of claim 10, wherein the pharmaceutical and/or dietary regimen comprises administration of a flavanol and/or a procyanidin oligomer and/or a derivative thereof.

12. The method of claim 10, wherein the condition is cardiovascular disease, coronary heart disease, cardiac fibrosis, atherosclerosis, and/or renal disease or failure.

13. A method of prophylactic or therapeutic treatment of a human or a veterinary animal comprising:
(i) determining ex vivo a baseline cytokine level in a body sample of the human or the veterinary animal, wherein the cytokine is associated with an inflammatory and/or immunomodulating pathway;
(ii) incubating the body sample with a series of flavanols and procyanidins and/or derivatives thereof, or a mixture thereof, under conditions sufficient to induce a change in cytokine levels, and measuring the resulting cytokine levels;
(iii) comparing the baseline cytokine level with the cytokine levels obtained in step (ii) to determine cytokine responsiveness of the human or the veterinary animal to flavanols and procyanidins and/or derivatives thereof;
(iv) designing, based on the cytokine responsiveness, a pharmaceutical and/or dietary regimen to promote homeostatic cytokine levels in the human or the veterinary animal; wherein the pharmaceutical and/or dietary regimen comprises administration of a flavanol and/or a procyanidin oligomer and/or a derivative thereof; and
(v) administering a flavanol and/or a procyanidin oligomer and/or a derivative thereof, or a mixture thereof, to the human or the veterinary animal according to the pharmaceutical and/or dietary regimen designed in step (iv).

14. A method of prophylactic or therapeutic treatment of a human or a veterinary animal comprising:
(i) determining ex vivo a baseline cytokine level in a body sample of the human or the veterinary animal, wherein the cytokine is associated with an inflammatory and/or immunomodulating pathway;
(ii) incubating the body sample with a series of flavanols and procyanidins and/or derivatives thereof, or a mixture thereof, under conditions sufficient to induce a change in cytokine levels, and measuring the resulting cytokine levels;
(iii) comparing the baseline cytokine level with the cytokine levels obtained in step (ii) to determine cytokine responsiveness of the human or the veterinary animal to flavanols and procyanidins and/or derivatives thereof;
(iv) diagnosing, based on the baseline cytokine level, whether the human or the veterinary animal is at risk of, or suffers from, a condition associated with inflammatory and/or immunomodulating pathways;
(v) designing a pharmaceutical and/or dietary regimen effective for prophylactic or therapeutic treatment of the condition diagnosed in step (iv); and
(vi) treating the human or the veterinary animal according to the pharmaceutical and/or dietary regimen designed in step (v).

15. The method of claim 14, wherein the pharmaceutical and/or dietary regimen comprises administration of a flavanol and/or a procyanidin oligomer and/or a derivative thereof.

16. The method of claim 14, wherein the condition is cardiovascular disease, coronary heart disease, cardiac fibrosis, atherosclerosis, and/or renal disease or failure.

17. The method of claims 1, 2, 5, 6, 9, 10, 13 or 14, wherein the cytokine is TGF-β.

18. The method of claims 1, 2, 5, 6, 9, 10, 13 or 14, wherein the cytokine is TGF-β1.

19. A screening assay for identifying cytokine responsiveness of a human or a veterinary animal comprising: (i) determining ex vivo a baseline cytokine level in a body sample of a human or a veterinary animal, wherein the cytokine is associated with an inflammatory and/or immunomodulating pathway; and (ii) incubating the body sample with a series of flavanols and procyanidins and/or derivatives thereof, or a mixture thereof, under conditions sufficient to induce a change in cytokine levels, and measuring the resulting cytokine levels.

20. The screening assay of claim 19, further comprising comparing the baseline cytokine level with the cytokine levels obtained in step (ii) to determine cytokine responsiveness of the human or the veterinary animal to flavanols and procyanidins and/or derivatives thereof.

21. The screening assay of claim 20, further comprising diagnosing the human or a veterinary animal as being at risk of, or suffering from, cardiovascular disease, coronary heart disease, cardiac fibrosis, atherosclerosis or renal disease or failure.

22. A method of determining a therapeutic value of a polyphenol for modulating cytokine levels in a mammal, the method comprising:
(i) obtaining body samples from at least one low cytokine producer and at least one high cytokine producer;
(ii) determining baseline cytokine levels in the body samples;
(iii) incubating the body samples with a polyphenol not known to have cytokine modulation properties under conditions sufficient to induce a change in cytokine levels;
(iv) determining the cytokine levels after the incubation of step (iii); and
(v) comparing the baseline cytokine levels with the cytokine levels of step (iv) to determine whether the polyphenol has cytokine modulating properties.

23. The method of claim 22, wherein the mammal is a human.

24. The method of claim 22, wherein the mammal is a veterinary animal.

25. A method of treating a subject, which is a low baseline TGF-β producer, comprising administering to the subject at least one flavanol and/or procyanidin oligomer selected from the group consisting of monomer, dimer, trimer, tetramer and pentamer, or any mixture or derivative thereof, in the amount effective to stimulate the level of TGF-β in the subject, wherein the subject is a human or a veterinary animal.

26. The method of claim 25, wherein TGF-β is TGF-β1.

27. A method of treating a subject, which is a high baseline TGF-β producer, comprising administering to the subject at least one procyanidin oligomer 6-10, or any mixture or derivative thereof, in the amount effective to stimulate the level of TGF-β in the subject, wherein the subject is a human or a veterinary animal.

28. The method of claim 27, wherein TGF-β is TGF-β1.
